(19) United States
(12) Patent Application Publication
(10) Pub. No.: US 2016/0287514 A1
(43) Pub. Date: Oct. 6, 2016

(54) MEDICINAL LOZENGE BASED ON IBUPROFEN SODIUM DIHYDRATE

(71) Applicant: PIERRE FABRE MEDICAMENT, Boulogne-Billancourt (FR)

(72) Inventors: Jean-François CORDOLIANI, SAINTE FOY D'AIGREFEUILLE (FR); Nadine HARAMBILLET, TOULOUSE (FR)

(73) Assignee: PIERRE FABRE MEDICAMENT, BOULOGNE-BILLANCOURT (FR)

(21) Appl. No.: 14/442,607

(22) PCT Filed: Nov. 14, 2013
(86) PCT No.: PCT/EP2013/073868

§ 371 Date: May 13, 2015

(30) Foreign Application Priority Data
Nov. 14, 2012 (FR) 1260815

(51) Int. Cl. A61K 9/00; A61K 45/06; A61K 31/192

(52) U.S. Cl. CPC A61K 9/0056; A61K 31/192; A61K 45/06

(57) ABSTRACT

The subject matter of the present invention is medicinal lozenges to be sucked, having a solid consistency so as to dissolve in the oral cavity, comprising at least ibuprofen sodium dihydrate as active ingredient, which are intended for the treatment of oropharyngeal conditions.

This invention relates to medicinal lozenges to be sucked, with a solid consistency, designed to dissolve in the oral cavity, comprising at least ibuprofen in the form of ibuprofen sodium dihydrate as active ingredient.

Inflammatory and painful disorders of the oropharyngeal area are incapacitating for patients and it is logical to conclude that the pharmacopeia is not helpful in providing fast, efficient and sufficiently long-lasting relief while limiting side effects. These oropharyngeal disorders have various origins and develop in the anterior part, on mucous membranes of the bottom and the sides of the mouth or on the posterior part on the pharyngeal mucous membrane. The oropharyngeal area is a constant and preferred access method for all germs and irritating substances brought in by air and food pathways. This zone is also a location for preferred development of bacterial populations, more or less pathogenic viruses justifying treatment of the inflammations that they cause. These inflammations may be more or less important and invalidating, varying from a simple sensation of local discomfort to the presence of macroscopically visible lesions of the type generated by oral aphthosis. There are often no major clinical signs such as fever or ganglion formations related to such inflammations.

Current treatments include the use of anti-inflammatory and/or antalgic products to be administrated locally: sprays, lozenges to be sucked, mouthwash. Available medicines are extremely limited since the disappearance of many products used as medicines, in other words compositions that have received a marketing authorisation. Thus products including combinations of enzymes, lysozyme, papain, contact anaesthetics and local antibiotics have lost their marketing authorisation. Products such as anaesthetics relieved pain rather than treating the cause and concealed the reality of the inflammation. One solution consists of making use of powerful anti-inflammatory drugs that reduce pain while also treating the associated inflammations. Such active ingredients are administrated through the digestive pathway with all the associated disadvantages.

Therefore the active ingredient has to be metabolised by the entire body thus inducing a generalised diffusion of the molecule in all organs and tissues. This broad diffusion is mostly useless because 100% of the body is treated in order to treat the 2% within the oropharyngeal area. Consequently, several problems arise that are not always easy to solve. The first is that a sufficient dose has to be administrated to the patient taking account of dilution and dispersion in the body, so that the significantly active part that reaches the affected zone is sufficiently effective. The second part concerns the latency time due to metabolisation and distribution in the body before the molecule acts on its target and the patient feels the benefits. The third difficulty is due to the consequences that such a massive diffusion of the active molecule might cause in the body, consequences that result in known side effects.

Non-steroidal anti-inflammatory drugs such as ibuprofen have been widely used for many years to treat acute pain. They act on mediators of the inflammation, namely tissular enzymes and particularly cyclo-oxigenases 1 and 2 and prostaglandines. Ibuprofen or 2-(4-(2-methylpropyl)phenyl)propanoic acid has been used for a long time as an analgesic in case of inflammation. Ibuprofen has many actions on different inflammatory pathways and cellular systems involved in acute and chronic inflammation. The main pharmacodynamic actions of ibuprofen and of other non-steroidal anti-inflammatory drugs are the implications in control of acute pain, fever and acute inflammatory reactions. Administration of ibuprofen in man for more than 40 years has made it possible to become very familiar with all the advantages and disadvantages of this non-steroidal anti-inflammatory drug and therefore it makes it an ideal candidate for use in another pharmaceutical form. Ibuprofen may be administrated in the form of strongly dosed tablets containing between 200 and 400 mg (Schachtel et al., 1994, Cli. Pharmacol. Ther., 55: 464-470). A recent meta-analysis reveals the significant superiority of ibuprofen (400 mg) compared with acetaminophene (1000 mg, paracetamol) for pain relief in pharyngitis (Frye et al., 2011, J. Fam. Pract., 60:293-294). Undesirable side effects are likely to occur at these relatively high doses. The main side effects of ibuprofen are gastritis, stomatitis, abdominal pains and even ulcers in the digestive tube, jaundice, headaches, tinnitus, sleepiness and confusion may also occur. Finally, allergic skin rashes and asthma may also be observed. Therefore it has been observed that side effects that are or that might be generated by absorption of such molecules can be worse than the pain and oropharyngeal inflammations, that are themselves locally invalidating.

Therefore there is a medical need to apply a local administration of an active molecule in an appropriate pharmaceutical form capable of acting as quickly as possible on oropharyngeal disorders and minimising undesirable effects. The choice has been made of a very well-known product, ibuprofen, to minimise safety problems. Another non-steroidal anti-inflammatory drug, flurbiprofen has already been described in this indication (WO 2006/092569). The shorter half-life of ibuprofen, about 2 hours compared with 4 hours for flurbiprofen, is advantageous for local treatment because it means that several doses may be taken during the day and therefore it provides more opportunities for relief of the patient and consequently better adaption of the dose to the demand.

Two pharmaceutical forms appear to be appropriate for local treatment, namely a lozenge to be sucked and medical tablets. A lozenge to be sucked is disclosed in many patent applications, for example based on ibuprofen lysinate (FR 2865648). However these lozenges to be sucked can create problems. They can be swallowed before being completely dissolved, and problems then arise due to the ingestion of medicine (choking, suffocation by obstruction of the throat). Moreover, these disadvantages may cause the treatment to be stopped. These problems are particularly important with the elderly and children. This is why the lozenge was chosen as the pharmaceutical form.

Medicinal lozenges to be sucked, also called boiled sugars are preparations based on sugary substances with a solid consistency intended to be dissolved in the mouth cavity. They may have a variety of forms, spherical, cylindrical, square, rectangular or polygonal. They are prepared from a syrup made from a diluting sugary substance brought to boiling point and then boiled at a higher temperature, typically from 100° C. to 160° C. Auxiliary substances are added to this sugary base such as sweeteners, antioxidants, colouring agents, flavours and the active ingredient(s). The active ingredients and auxiliary substances are added to the mass in a mixer during boiling or during cooling. The mass thus prepared is kneaded on an appropriate cold surface and is then rolled and formed into a strip before it is pressed and cut into lozenges with the required shape and dimensions.

These medicinal lozenges to be sucked are made from boiled sugar and are essentially intended for local treatment of the mouth and oropharyngian area due to the location in which they are dissolved, but can also be used as active ingredients absorbed perlingually. Consequently, the active ingredients chosen for these action modes must be released progressively to remain in contact with the oropharyngeal sphere for as long as possible, while avoiding fast and massive passage in the digestive tract which would have the consequence of making them less efficient at the application location or the chosen absorption pathway. In general, medicinal lozenges made from boiled sugar dissolve in 10 minutes on average.

According to one embodiment of the invention, a matrix agent may be added to the composition in order to slow release of the active ingredient(s). The dissolution time of the lozenge in the oral cavity is then at least 15 minutes. Furthermore, the matrix agent confers increased resistance on the lozenge, making this resistance durable even when the lozenge is in contact with saliva such that the patient cannot crunch the lozenge and swallow the pieces.

The matrix agent is chosen from the group composed of non-cellulosic polysaccharides, cellulose derivatives, acrylic acid polymers, fatty substances and polyvinylpyrrolidone, these substances being used alone or mixed together and representing 1 to 10% by weight of the lozenge.

According to one embodiment, the matrix agent is chosen from the group consisting of guar gum, locust bean gum, sodium and potassium alginate, agar agar, carrageenan, arabic gum, sterculia gum, tragacanth.

According to one embodiment, the matrix agent is chosen from the group composed of hydroxypropylcellulose, hydroxypropylmethylcellulose, hydroxyethylcellulose, ethylcellulose.

According to one embodiment, the acrylic acid polymer is a carbomer or a polymethacrylate, vinyl acetate copolymer.

According to one embodiment, the fatty substance is chosen from among the group composed of waxes, gelucires, glyceryl behenate, glyceryl palmitostearate.

Medicinal lozenges made from boiled sugar are composed of a large proportion of a sugary diluting substance or excipient forming the base of the preparation that may be saccharose, fructose, lactose, maltose, sorbitol, mannitol, lactitol, glucose, maltitol, isomalt, polydextrose, maltodextrins, used alone or mixed together, and that represents 80 to 99% of the lozenge by weight.

According to one embodiment, the lozenge also comprises at least one auxiliary substance chosen from among sweeteners, antioxidants, colouring agents, flavours.

One or several sweeteners may be chosen from among the group composed of acesulfame, aspartame, cyclamic acid and its salts, isomalt, saccharine and its salts, sucralose, alitame, thaumatin, glycyrrhizic acid and its salts, neohesperidin dihydrochalcone, steviol glucosides, neotame, aspartame-acesulfame salt, tagatose, polyglycitol syrup, maltitol, maltitol syrup, lactitol, xylitol, erythritol.

One or several antioxidants may be chosen from among the group composed of ascorbic acid, sodium ascorbate, calcium ascorbate, ascorbyl diacetate, ascorbyl palmitate, ascorbyl stearate, different tocopherols, gallates, guaiacum resin, erythorbic acid, sodium erythorbate, potassium or calcium isoascorbate, butylhydroquinone, butylhydroxyanisol, butylhydroxytoluene.

Colouring agents and flavours may be natural and/or artificial, and are well known to those skilled in the art.

The method of manufacturing medicinal lozenges comprises four steps, namely a boiling step, a cooking step, a mixing step and a step to fabricate the boiled sugar lozenges. The fabrication method complies with existing pharmaceutical requirements. The galenic lozenge form is very specific, and a monograph has been produced for it in the French Pharmacopeia. The lozenges are solid saccharoids that will slowly disintegrate in the mouth. In particular they are hemispherical in shape and usually weigh between 1 g and 3 g.

For the purposes of this invention, an oropharyngeal disorder includes all sore throats, particularly acute sore throats, the causes being viral in most cases, in other words pharyngeal pains caused by inflammation in the oral cavity, in the larynx and/or pharynx. A reddish throat associated with difficulty in swallowing are classical symptoms of a sore throat. It should also include pharangitis, in other words an inflammation of the pharynx and tonsils, rhinopharyngitis that is an inflammation of the upper stage of the pharynx, allergies such as rhinitis that can cause itching of the palate, laryngitis, acute inflammation of the larynx and also stomatitis, aphtha and gingivitis.

This invention discloses new solid medicinal lozenges made from boiled sugar based on ibuprofen. They can be used for local treatment of pathologies in the oral area and to release the active ingredient on the surface of the oropharyngeal zone to be treated, while having a dose slightly less than that in existing pharmaceutical forms.

However, a technical problem arose, namely that the melting temperature of ibuprofen in its basic form is of the order of 72° C. But it has already been mentioned that the temperature that has to be reached for preparation of these lozenges is well above 100° C. and possibly as high as 160° C. The use of basic ibuprofen in molten form makes it difficult to homogenise the active ingredient in the substance and causes fast oxidation of ibuprofen, to the extent that the taste becomes terrible. Therefore the use of basic ibuprofen is incompatible with the fabrication method for these medicinal lozenges.

Application WO2006/092569 discloses medicinal lozenges to be sucked made of boiled sugar comprising a non-steroidal anti-inflammatory substance, mainly flurbiprofen for their use to treat sore throats. The disclosed invention concerns a new method to produce a pharmaceutical formulation for lozenges, including particularly a step to a obtain a liquid composition comprising a salt of a non-steroidal anti-inflammatory agent. Examples of the preparation of liquid compositions are reported in the patent application. Many different liquid compositions containing flurbiprofen are thus described, some examples containing ibuprofen in the form of sodium salt or potassium salt are also mentioned. However, stability studies of these liquid compositions only mention flurbiprofen.

This invention aims at making use of an ibuprofen salt in order to obtain better resistance to a temperature increase. Three salts can be used by this method, namely arginine, lysinate and sodium dihydrate.

Knowledge of the behaviour of hydration and dehydration of drugs is fundamental to develop stable pharmaceutical formulations and to obtain appropriate storage conditions. The authors of a study on sodium ibuprofen hydration and dehydration mechanisms conclude that ibuprofen in the form of sodium dihydrate salt is the most stable form (Censi et al., 2013, J. Therm Anal Calorim, 111; 2009-2018), which explains why sodium salt dihydrate was preferred.

Analytic studies done on three batches of lozenges containing one of the three possible ibuprofen salts surprisingly concluded that only ibuprofen sodium dihydrate satisfied all criteria for pharmaceutical development.

Therefore this invention applies to solid medicinal lozenges to be sucked made from boiled sugar designed to dissolve in the mouth, containing ibuprofen sodium dihydrate as its active ingredient (CAS-No. 31121-93-4).

According to the invention, the dose of ibuprofen per lozenge is between 5 and 50 mg (equivalent to 6.4 and 64 mg of ibuprofen sodium dihydrate).

Preferably, the dose of ibuprofen per lozenge is 15 mg (equivalent to 19.2 mg of ibuprofen sodium dihydrate).

Also preferably, the dose of ibuprofen per lozenge is 25 mg (equivalent to 32 mg of ibuprofen sodium dihydrate).

Also preferably, the dose of ibuprofen per lozenge is 35 mg (equivalent to 44.8 mg of ibuprofen sodium dihydrate).

In yet another embodiment of the invention, the medicinal lozenge based on ibuprofen sodium dihydrate will be used for adults or children more than 12 years old.

According to another embodiment, the medicinal lozenge based on ibuprofen sodium dihydrate is intended for children more than 6 years old. A pharmacological safety profile was produced for a child and this pharmaceutical form, namely a medicinal lozenge made of boiled sugar, may be used for children more than 6 years old.

According to another embodiment of the invention, the medicinal lozenge based on ibuprofen sodium dihydrate also comprises at least one other active ingredient useful in oropharyngeal disorders.

Another active ingredient means an antalgic, a non-steroidal anti-inflammatory agent, a local anaesthetic, an antiseptic, a local antibacterial agent, or a corticoid for local use.

This invention also relates to the use of a medicinal lozenge based on ibuprofen sodium dihydrate for the preparation of a medicine for the treatment of oropharyngeal disorders.

An example of an analytic study is given below, which highlights the choice of ibuprofen salt.

### EXAMPLE 1

### Analysis of Impurities

The ibuprofen stability study was carried out on 2.5 g lozenges containing ibuprofen arginine, sodium or lysinate salts. Different preservation conditions have been tested, the temperature (25 or 40° C.), the relative humidity (RH) 60 or 75%, these tests were done at T0, 15 days, 1, 2 and 3 months.

The total content of impurities was measured.

TABLE 1
(values as percentage)

| | Conditions | | | | | |
|---|---|---|---|---|---|---|
| | | | Pill | | | |
| | | | box | Salts | | |
| | T° C. | RH | cond. | Arg | Sod | Lys |
| T0 | | | C | <0.1 | <0.1 | <0.1 |
| T15 d | 40 | 75 | C | 0.72 | <0.1 | 0.28 |
| T1 month | 25 | 60 | C | 0.15 | <0.1 | 0.15 |
| | 40 | 75 | C | 0.24 | <0.1 | 0.19 |
| | 40 | 75 | O | 1.66 | <0.1 | 0.53 |
| T2 months | 25 | 60 | C | 0.25 | <0.1 | 0.15 |
| | 40 | 75 | C | 0.48 | <0.1 | 0.21 |
| | 40 | 75 | O | 2.94 | <0.18 | 2.21 |
| T3 months | 25 | 60 | C | 0.29 | <0.1 | 0.17 |
| | 40 | 75 | C | 0.63 | 0.16 | 0.27 |
| | 40 | 75 | O | 3.86 | 0.40 | 3.10 |

T0 is the base time,
T15 d: at 15 days
T° C. = temperature,
RH = relative humidity;
Co pi = pill box conditioning;
C: closed pill box;
O: open pill box;
Arg: arginine;
Sod: sodium,
Lys: lysinate
Underlined values represent the presence of significant quantities of impurities higher than 0.25% relative to the initial content.

It is found that the composition with the lowest content of impurities regardless of the long term temperature and humidity conditions is sodium dihydrate salt (table 1). The form that is least degraded under specific temperature and humidity conditions is sodium dihydrate salt with a percent of impurities that does not vary significantly, unlike arginine and lysinate salts.

### EXAMPLE 2

### Composition of the Lozenge According to the Invention

| | |
|---|---|
| Ibuprofen sodium dihydrate* | 19.2 mg equivalent to |
| | 15 mg of ibuprofen |
| Ascorbic acid | 50.0 mg |
| Hydroxypropylmethylcellulose | 100.0 mg |
| Saccharose | 1325.0 mg |
| Glucose | 1000.0 mg |
| Flavour | QSP |

### EXAMPLE 3

### Composition of a Lozenge According to the Invention

| | |
|---|---|
| Ibuprofen sodium dihydrate* | 32 mg equivalent to |
| | 25 mg of ibuprofen |
| Isomalt | 2417.0 mg |
| Potassium acesulfame | 15.0 mg |
| Sucralose | 11.0 mg |
| Flavour | QSP |

### EXAMPLE 4

### Composition of a Lozenge According to the Invention

| | |
|---|---|
| Ibuprofen sodium dihydrate* | 44.8 mg equivalent to |
| | 35 mg of ibuprofen |
| Chlorhexidine digluconate | 3.0 mg |
| Ascorbic acid | 52.5 mg |
| Maltitol | 2408.0 mg |
| Aspartame | 1.0 mg |
| Cochineal red | 0.2 mg |
| Flavour | QSP |
| Water | QSP |

*For example marketed by the BASF Company

### EXAMPLE 5

### Clinical Study

An effectiveness and security study is carried out to compare tablets according to the invention with a placebo for the treatment of persons suffering from acute sore throats.

The main purpose is to compare the effect of lozenges according to the invention dosed at 25 mg of ibuprofen in comparison with the placebo on total relief of pain over a period of 2 hours, after the first administration of the product.

Secondary objectives are also fixed, like evaluation of pain relief over different measurement times, evaluation of the beginning of the effect versus a placebo and finally the local and global tolerance of lozenges according to the invention versus a placebo, in repeated doses. It is a multi-centric, randomised study versus a placebo on 2 parallel groups in 2 phases.

A stationary phase in the investigation centre with an evaluation made at 15 minutes, 30 minutes, 45 minutes, 60 minutes, 90 minutes and 120 minutes after starting sucking the lozenge in the first administration of the product being studied.

An ambulatory phase from D1 to D4.

Finally, an end of study visit is planned for D5/D6.

Clinical evaluations of effectiveness and tolerance parameters take place every 30 minutes until 120 minutes after starting sucking, and then every 60 minutes until 360 minutes. The study is performed in specialised centres with long experience with sore throats. A total of 385 patients were included from 4 countries.

The result of the main criterion of total pain relief after 2 hours is favourable with a statistically significant effect in favour of lozenges according to the invention dosed with 25 mg of ibuprofen, versus a placebo (p=0.045).

The results of this main criterion for other measurement times are also in favour of lozenges according to the invention with a statistically significant difference,

15 min: p=0.012;

30 min: p=0.007;

45 min: p=0.005;

60 min: p=0.007;

90 min: p=0.012.

The difference in the effect on total pain relief over a period of 2 hours is +0.44 in favour of lozenges according to the invention, namely +14% total pain relief versus a placebo. This difference in effect between the 2 groups is fast and maximum at the beginning of the treatment with about +30% relief in the group of lozenges according to the invention dosed at 25 mg of ibuprofen versus a placebo, within 15 minutes after administration.

Finally, no difference in tolerance is observed between the 2 groups, illustrating optimum security for lozenges according to the invention.

In conclusion, this clinical study demonstrates that lozenges according to the invention are effective and safe for the treatment of acute sore throats.

1-14. (canceled)

15. Medicinal lozenge to be sucked made from boiled sugar, with a solid consistency, designed to dissolve in the oral cavity, comprising ibuprofen in the form of ibuprofen sodium dihydrate as active ingredient.

16. Lozenge according to claim 15, wherein the dose of ibuprofen per lozenge is between 5 and 50 mg (equivalent to 6.4 and 64 mg of ibuprofen sodium dihydrate).

17. Lozenge according to claim 16, wherein the dose of ibuprofen per lozenge is 15 mg (equivalent to 19.2 mg of ibuprofen sodium dihydrate).

18. Lozenge according to claim 16, wherein the dose of ibuprofen per lozenge is 25 mg (equivalent to 32 mg of ibuprofen sodium dihydrate).

19. Lozenge according to claim 16, wherein the dose of ibuprofen per lozenge is 35 mg (equivalent to 44.8 mg of ibuprofen sodium dihydrate).

20. Lozenge according to claim 15, wherein ibuprofen sodium dihydrate is associated with at least one other active ingredient useful in oropharyngeal disorders.

21. Lozenge according to claim 15, comprising at least 1 to 10%, by weight of the lozenge, of at least one matrix agent.

22. Lozenge according to claim 21, wherein the at least one matrix agent is chosen from among non-cellulosic polysaccharides, cellulose derivatives, acrylic acid polymers, fatty substances and polyvinylpyrrolidone, or a combination of them.

23. Method of preparing a medicinal lozenge according to claim 15, comprising the following steps:
a) boiling a syrup made from a diluting sugary substance;
b) cooking the preparation in step a);
c) mixing the preparation obtained in step b) with the active ingredient(s) and at least one auxiliary substance chosen from among sweeteners, antioxidants, matrix agents, colouring agents, and flavours; then;
d) fabricating the boiled sugar lozenges.

24. Method according to claim 23, wherein step b) is carried out at a temperature between 100° C. and 160° C.

25. Method according to claim 23, wherein said syrup made from a diluting sugary substance is chosen from among saccharose, fructose, lactose, maltose, sorbitol, mannitol, lactitol, glucose, maltitol, isomalt, polydextrose, maltodextrins or combination of them.

26. Method according to claim 24, wherein the sweetener is chosen from among acesulfame, aspartame, cyclamic acid and its salts, isomalt, saccharine and its salts, sucralose, alitame, thaumatin, glycyrrhizic acid and its salts, neohesperidin dihydrochalcone, steviol glucosides, neotame, aspartame-acesulfame salt, tagatose, polyglycitol syrup, maltitol, maltitol syrup, lactitol, xylitol, erythritol.

27. Method of treating oropharyngeal disorders which comprises administering to a patient lozenges according to claim 15.

28. Method according to claim 27 wherein the oropharyngeal disorder is acute sore throats.

29. Method according to claim 27 wherein the patient is an adult or child more than 12 years old.

30. Method according to claim 27, wherein the patient is a child more than six years old.
